SEQUENCE LISTING (7 sequences)

SEQ ID NO: 1 - DNA, 7184 bp, Homo sapiens
cattcataagactcagagctacggccacggcagggacacgcggaaccaagacttggaaacttgattgttgtggttcttcttgggggttatgaaatttcattaatctttttttttccggggagaaagtttttggaaagattcttccagatatttcttcattttcttttggaggaccgacttactttttttggtcttctttattactcccctccccccgtgggacccgccggacgcgtggaggagaccgtagctgaagctgattctgtacagcgggacagcgctttctgcccctgggggagcaacccctccctcgcccctgggtcctacggagcctgcactttcaagaggtacagcggcatcctgtgggggcctgggcaccgcaggaagactgcacagaaactttgccattgttggaacgggacgttgctccttccccgagcttccccggacagcgtactttgaggactcgctcagctcaccggggactcccacggctcaccccggacttgcaccttacttccccaacccggccatagccttggcttcccggcgacctcagcgtggtcacaggggcccccctgtgcccagggaaatgtttcaggctttccccggagactacgactccggctcccggtgcagctcctcaccctctgccgagtctcaatatctgtcttcggtggactccttcggcagtccacccaccgccgccgcctcccaggtaagtttttgatagtaggggtgctgctttgtaggttttattttttaagtcaagggtgaaaagaataaaccccaacccccacaaaaaggcgcatcagaaccctagatctgagatggaaaaggctcacagcgcactttgcaaactgcaaagagtcgggagatgtttgcaattggttgcgtgcgtggagcgcaaggagggaacgcggcagggagggtaggctttggggcgaggtgggggtggggtgggtaatgcgctgctcaatgcaacgtgtatgcggtagcggggctgagaactttgagccggccccgggactgccccctgctcgggtcccagacctgaagctagcgcagttaggcaggtgggggaaatcccggggaagcttccagcagtctcttttcctttcctcctccttcggagcgcccacttcggtgccgggtcgccctccacccatcgggaagaggggcctcgaaccctcagccgcgctgcctccgcctcctgcgcggagacgtaacgggggacccgtgcgtaacggctgacgcgctggaatcctccgtctgacgcggggcacgcacggcgcgcggcgcccccttcgtccgccccgcccctgacgtcccgggagcgttctattttggaacgccggggccacgttgctaagggagggggcagcccggcgtttcgattggccgccggggcgcacgccttggccaatcagctttcccttcctatttgtagggtgcattttccttcccccctctctgtccccggaacccgtggttccttggcggctgggtctcttttcggcgcctctagaggcagagggaggggatccctgtcgtgacaagagcgcctgtctgcgacccaatggatctgcgaggcccttgcggggatctagtccctgggctctcaggagaagggggtgtctgcttgtgtgctggcgtttcttggagagatacggtggctgtcaccctcttctccaggcacacacagacacattcccactccctctgctcctcatgcccggttcctccggtgtgtcccaagacaggactagaaccgcgaaccgaagggcaaccagccaggtggtctccaggagctccgccccctctgggttcccaggactctgattggtcggcgagccagcccttccctcaccacgccccccgagagagtagttaagccttcagagcagttccaggagtccatttacgggaggggggagatgagcgctgctgaggcttgggggctcaggtcccgcaccattccccctccgcgacaatctgagagagctccagtggttacttttatctacctgtccgttcaccctaaactgtcactcgtcagtctcactctgagaagagacagtaacttgaaacgttgttctaaactcctaggcccgtcccccaaacacccttttgactgggacccccgcccctgcatgggacctcgcgcagaggggggtgtgtatgtgtgtgagtgtagaggaaggcttggcctaaggcctctccttctccctccccttgcctctggggtgggggtggggtgttgtggctgtgtgtgtggctgtggctccgtcccgggggttctgtcacccggctgtgtccagcctcctctccaccccccatacctaagagtcaccaacccggggtgtgattcaccacccgctggaaccgtgcaacctttccccgaggaagaaggaggaggtagaagccagttgagcagaaatcctctcattaaccactgcgtcacggtgtagtggaagggtgggtgttgtggctttttccctgtgacacacacatccacactcgctcaccctgtgctcactcacggggtcggtgtgtgttatgtgtgttgggtgtgtgtgtgtcggtgtctttgtttgtgtgtctacgcctgtgtgtgtatgtgtcaccccgtaggagtgcgccggtctcggggaaatgcccggttccttcgtgcccacggtcaccgcgatcacaaccagccaggacctccagtggcttgtgcaacccaccctcatctcttccatggcccagtcccaggggcagccactggcctcccagcccccggtcgtcgacccctacgacatgccgggaaccagctactccacaccaggcatgagtggctacagcagtggcggagcgagtggcagtggtgggccttccaccagcggaactaccagtgggcctgggcctgcccgcccagcccgagcccggcctaggagaccccgagaggagacggtgagtaagggacatcagaacttggcctgggtagggggaagcaagagaggcaggaagtttcttatgaatggaggggggctccactaaggcctcagtgttacagaaaccccaagatccttgctacacgagcgaggaccggaggcttgttttttggctcttgggggttctgaaagaagtagaggttcgggatgggtggaggagtgctgtatccccaaatctcatggcctctatctccctgactcagctcaccccagaggaagaggagaagcgaagggtgcgccgggaacgaaataaactagcagcagctaaatgcaggaaccggcggagggagctgaccgaccgactccaggcggtgaggacaggccctggggtgggagaggggatgttgaggggagctctctccccattctctgccccctctccacctgtacccttatcctgggttgagaactagacgttccacacatggaactaggtactgctgtggccagactgggtagcccagggcacaaacacagacccccatggacttaagtcaactcctggtcccccccccatttcctgaccccacggactactctcctagccttcatttcatcccaaggggccacatggggcccttgaggagaggggcgcccccatcatttctcccatctggtcttcagcaagtaacaacccattttgcctcagtttctccatctctgcaaaccctcatcaaatctcccgggctctttctacccttaacactctggaaagcctgtgaaatgaaattattccacctcctgccctagccacccacagctctcctggtgctggtggcatcccccaaaacccactcccttcctacgtcctcccttggtctgagagttccctgctgtatgcctgcagggtgagctgttactccttgagggaacaagggaattgtcaactttccttctctactttttctcttccccgggaggtagagagggaggggtaatagaagggaacacattaaaaacacataacagtggctcatgcctgtgatcccagcactttgagaggccaaggcaggaggattgcttgagcccaggagtttgagaccagcctgggaaacatagggaggacttgtctctaccaagaaaaaaaaaaattagttgggcatggtggtgcacaccactgtggtcccagctactatggaggctttagtgggaggatcgcttgagccgaggaggtccaggctgcagtgagccatgattgcactattgcactccagcctgggggacagagcgagactctggctcaaaagcaaaacaaaaccaaccacataacgattgttcattcattcaacaaacctcctgcacaccagaaacttccccataaacctggccccttctgtaccatcctttggacagatgggggaaactgaggttcctggaaggcaagacccttgccccaactcccatggccaccaggactccatctcaggaggtgttttttctcagcccggggctgccttccagcagcaagtccaagggagccatgacccgagtttcccggtcactgacatgcttttttctccttcctctctctcttctgtgacctggcctccctggcctcaggagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggaggtgagagatttgccgggctcagcaccggctaaggaagatggcttcagctggctgctgccgcccccgccaccaccgcccctgcccttccagaccagccaagacgcaccccccaacctgacggcttctctctttacacacagtgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctctgtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggattaagggacgggggtgggaggtaggctgtggggtgggctggagtcctctccagagaggctcaacaaggaaaaatgccactccctacccaatgtctcccacacccaccctttttttggggtgcctaggttggtttcccctgcactcccgaccttagcttattgatcccacatttccatggtgtgagatcctctttactctgggcagaagtgagccccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaagggagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgc

SEQ ID NO: 2 - DNA, 156 bp, Homo sapiens
gagacagatcagttggaggaagaaaaagcagagctggagtcggagatcgccgagctccaaaaggagaaggaacgtctggagtttgtgctggtggcccacaaaccgggctgcaagatcccctacgaagaggggcccgggccgggcccgctggcggag

SEQ ID NO: 3 - DNA, 2332 bp, Homo sapiens
tgaagttcaagtcctcggcgaccccttccccgttgttaacccttcgtacacttcttcgtttgtcctcacctgcccggaggtctccgcgttcgccggcgcccaacgcaccagcggcagtgaccagccttccgatcccctgaactcgccctccctcctcgctctgtgaactctttagacacacaaaacaaacaaacacatgggggagagagacttggaagaggaggaggaggaggagaaggaggagagagaggggaagagacaaagtgggtgtgtggcctccctggctcctccgtctgaccctctgcggccactgcgccactgccatcggacaggaggattccttgtgttttgtcctgcctcttgtttctgtgccccggcgaggccggagagctggtgactttggggacagggggtgggaaggggatggacacccccagctgactgttggctctctgacgtcaacccaagctctggggatgggtggggaggggggcgggtgacgcccaccttcgggcagtcctgtgtgaggattaagggacgggggtgggaggtaggctgtggggtgggctggagtcctctccagagaggctcaacaaggaaaaatgccactccctacccaatgtctcccacacccaccctttttttggggtgcctaggttggtttcccctgcactcccgaccttagcttattgatcccacatttccatggtgtgagatcctctttactctgggcagaagtgagccccccccttaaagggaattcgatgcccccctagaataatctcatccccccacccgacttcttttgaaatgtgaacgtccttccttgactgtctagccactccctcccagaaaaactggctctgattggaatttctggcctcctaaggctccccaccccgaaatcagcccccagccttgtttctgatgacagtgttatcccaagaccctgccccctgccagccgaccctcctggccttcctcgttgggccgctctgatttcaggcagcaggggctgctgtgatgccgtcctgctggagtgatttatactgtgaaatgagttggccagattgtggggtgcagctgggtggggcagcacacctctggggggataatgtccccactcccgaaagcctttcctcggtctcccttccgtccatcccccttcttcctcccctcaacagtgagttagactcaagggggtgacagaaccgagaagggggtgacagtcctccatccacgtggcctctctctctctcctcaggaccctcagccctggcctttttctttaaggtcccccgaccaatccccagcctaggacgccaacttctcccaccccttggcccctcacatcctctccaggaagggagtgaggggctgtgacatttttccggagaagatttcagagctgaggctttggtacccccaaacccccaatatttttggactggcagactcaaggggctggaatctcatgattccatgcccgagtccgcccatccctgaccatggttttggctctcccaccccgccgttccctgcgcttcatctcatgaggatttctttatgaggcaaatttatattttttaatatcggggggtggaccacgccgccctccatccgtgctgcatgaaaaacattccacgtgccccttgtcgcgcgtctcccatcctgatcccagacccattccttagctatttatccctttcctggtttccgaaaggcaattatatctattatgtataagtaaatatattatatatggatgtgtgtgtgtgcgtgcgcgtgagtgtgtgagcgcttctgcagcctcggcctaggtcacgttggccctcaaagcgagccgttgaattggaaactgcttctagaaactctggctcagcctgtctcgggctgacccttttctgatcgtctcggcccctctgattgttcccgatggtctctctccctctgtcttttctcctccgcctgtgtccatctgaccgttttcacttgtctcctttctgactgtccctgccaatgctccagctgtcgtctgactctgggttcgttggggacatgagattttattttttgtgagtgagactgagggatcgtagatttttacaatctgtatctttgacaattctgggtgcgagtgtgagagtgtgagcagggcttgctcctgccaaccacaattcaatgaatccccgacccccctaccccatgctgtacttgtggttctctttttgtattttgcatctgaccccggggggctgggacagattggcaatgggccgtcccctctccccttggttctgcactgttgccaataaaaagctcttaaaaacgc

SEQ ID NO: 4 - DNA, 20 bp, Artificial Sequence
  Description of Artificial Sequence Synthetic primer
aaaaggcagagctggagtcg

SEQ ID NO: 5 - DNA, 20 bp, Artificial Sequence
  Description of Artificial Sequence Synthetic primer
gtacgaagggctaacaacgg

SEQ ID NO: 6 - DNA, 18 bp, Artificial Sequence
  Description of Artificial Sequence Synthetic primer
aaagcagagctggagtcg

SEQ ID NO: 7 - DNA, 20 bp, Artificial Sequence
  Description of Artificial Sequence Synthetic primer
gtacgaagggttaacaacgg

(19) United States
(12) Patent Application Publication
(10) Pub. No.: US 2006/0057624 A1
(43) Pub. Date: Mar. 16, 2006

(54) Alternatively spliced pre-mRNA transcripts in neurodegenerative disease

(76) Inventor: Judith Ann Potashkin, Gurnee, IL (US)

Correspondence Address: EVEREST INTELLECTUAL PROPERTY LAW GROUP, P.O. Box 708, Northbrook, IL, 60065, US

(21) Appl. No.: 11/215,842

(22) Filed: Aug. 30, 2005

Related U.S. Application Data
(60) Provisional application No. 60/605,643, filed on Aug. 30, 2004.

(51) Int. Cl. C12Q 1/68

(52) U.S. Cl. 435/6

(57) ABSTRACT

The present invention provides a method of diagnosing a neurodegenerative disease in a mammalian subject, preferably a human subject. The method comprises obtaining RNA from the mammalian subject, and assaying the RNA for an increase in the amount of ΔFosB mRNA or in ratio of the amount of ΔFosB mRNA to the amount of FosB mRNA as compared to that of a control. An increase in the amount of ΔFosB mRNA or the ratio of the amount of ΔFosB mRNA to the amount of FosB mRNA as compared to that of the control is indicative of the presence of the neurodegenerative disease.

### CROSS-REFERENCE TO RELATED APPLICATIONS

This application claims priority from provisional application Ser. No. 60/605,643 filed Aug. 30, 2004, which is incorporated herein by reference and made a part hereof.

### FEDERALLY SPONSORED RESEARCH OR DEVELOPMENT:

Not Applicable.

### TECHNICAL FIELD

The present invention provides a method of diagnosing or prognosticating a neurodegenerative disease in a mammalian subject, preferably human.

### REFERENCE TO SEQUENCE LISTING

A sequence listing is included as a part of this disclosure and all information contained therein is incorporated herein by reference.

### BACKGROUND OF THE INVENTION

Neurodegenerative diseases can be quite debilitating. Parkinson's disease, for example, is characterized by the progressive and selective loss of nigrostriatal dopamine (DA) neurons. Most cases of Parkinson's disease are sporadic, suggesting a strong environmental influence. The molecular basis of idiopathic Parkinson's disease remains unknown. Alzheimer's disease is also characterized by the progressive loss of brain cells.

In view of the foregoing, it would be beneficial to have a method of diagnosing neurogenerative disease, such as Parkinson's disease and Alzheimer's disease, as early as possible. In this regard, it would be beneficial to be able to monitor the progression of disease as well as the efficacy of treatment. It is an object of the present invention to provide such methods. This and other objects and advantages of the present invention, as well as additional inventive features, will become apparent from the detailed description provided.

### BRIEF SUMMARY OF THE INVENTION

The present invention provides a method of diagnosing a neurodegenerative disease in a mammalian subject, preferably a human subject. The method comprises obtaining RNA from the mammalian subject, and assaying the RNA for an increase in the amount of ΔFosB mRNA or in the ratio of the amount of ΔFosB mRNA to the amount of FosB mRNA as compared to that of a control. An increase in the amount of ΔFosB mRNA or the ratio of the amount of ΔFosB mRNA to the amount of FosB mRNA as compared to that of the control is indicative of the presence of the neurodegenerative disease.

The present invention further provides a method of prognosticating a neurodegenerative disease in mammalian subject, preferably a human subject. The method comprises obtaining RNA from the subject over time, and assaying the RNA to obtain the amount of ΔFosB mRNA and the amount of FosB mRNA, or the ratio of the amount of ΔFosB mRNA to the amount of FosB mRNA. An increase in the amount of ΔFosB mRNA or the ratio of the amount of ΔFosB mRNA to the amount of FosB mRNA is indicative of a negative prognosis, whereas no change in the amount of ΔFosB mRNA or the ratio of the amount of ΔFosB mRNA to the amount of FosB mRNA or a decrease in the amount of ΔFosB mRNA or the ratio of the amount of ΔFosB mRNA to the amount of FosB mRNA is indicative of a positive prognosis.

The present invention still further provides a method of assessing the efficacy of treatment of a neurodegenerative disease in a mammalian subject in need of the treatment. The subject is preferably a human subject. The method comprises obtaining RNA from the subject during the course of treatment, and assaying the RNA to obtain the amount of ΔFosB mRNA or the ratio of the amount of ΔFosB mRNA to the amount of FosB mRNA. An increase in the amount of ΔFosB mRNA or the ratio of the amount of ΔFosB mRNA to the amount of FosB RNA indicates that the treatment is ineffective, whereas no change in the amount of ΔFosB mRNA or the ratio of the amount of ΔFosB mRNA to the amount of FosB mRNA or a decrease in the amount of ΔFosB mRNA or the ratio of the amount of ΔFosB mRNA to the amount of FosB mRNA indicates that the treatment is effective.

The present invention yet further provides a method of screening for potential therapeutic agents for the treatment of a neurodegenerative disease in a mammalian subject in need of the treatment. The subject is preferably a human subject. The method comprises obtaining RNA from the subject after and/or during the course of the administration of the agent, and assaying the RNA to obtain the amount of the ΔFosB mRNA or the ratio of the amount of ΔFosB mRNA to the amount of FosB mRNA. An increase in the amount of ΔFosB mRNA or the ratio of the amount of ΔFosB mRNA to the amount of FosB mRNA indicates that the therapeutic agent being screened is a potential therapeutic agent for the disease, whereas no change in the ratio of the amount of ΔFosB mRNA to the amount of FosB RNA or a decrease in the ratio of the amount of ΔFosB mRNA to the amount of FosB mRNA indicates that the therapeutic agent being screened is not a potential therapeutic agent for the disease.

In view of the above, the present invention also provides a kit for use in such methods. The kit comprises a sense primer from exon 4 of the human or mouse FosB gene and an antisense primer from exon 5 of the human or mouse FosB gene. The human primers are preferred for use in analyzing samples obtained from humans.

### BRIEF DESCRIPTION OF THE DRAWINGS

FIG. 1 is a schematic diagram indicating the alternative splicing of FosB pre-mRNA to form ΔFosB and FosB mRNA (E1=exon 1, E2=exon 2, E3=exon 3, E4=exon 4, and E5=exon 5);

FIG. 2 shows the representative images of RT-PCR amplification of synphilin, FosB and GABAB1 receptor RNA variants (S.N.=Substantia nigra; Str.=Striatum; P=Probenecid; M=MPTP/probenecid; Synphilin (L)=large synphilin; Synphilin; (S)=small synphilin; 1a=GABAB1a; and 1b=GABAB1b);

FIG. 3 shows the ratio of synphilin (S) to synphilin (L) increased in substantia nigra after MPTP/Probenecid treatment. (S=short; L=long;+indicates p<0.05);

FIG. 4 shows the ratio of GABAB1b to GABAB1a decreased in the striatum of MPTP/Probenecid-treated mice; # indicates P<0.05);

FIG. 5 shows that ΔFosB protein increased slightly in the striatum of mice after MPTP/Probenecid treatment using actin as the loading control († means P=0.1);

FIG. 6 shows the increase in the ratio of ΔFosB mRNA to FosB mRNA in mice striatum after chronic MPTP/probenecid treatment (* indicates P<0.05).

### DETAILED DESCRIPTION OF THE INVENTION

While this invention is susceptible to embodiments in many different forms, it is shown in the drawings and will herein be described in detail a preferred embodiment of the invention with the understanding that the present disclosure is to be considered as an exemplification of the principles of the invention and is not intended to limit the broad aspect of the invention to the embodiments illustrated.

The present invention provides a method of diagnosing or prognosticating neurodegenerative diseases in a mammalian subject, preferably human, by assaying alternatively spliced precursor-messenger RNA (pre-mRNA) transcripts in tissues (e.g., blood or cerebrospinal fluid) obtained from the subject.

There is no cure for these neurodegenerative diseases and, to date, there are no biomarkers to identify susceptible individuals. With new tools, such as those disclosed in the present invention, i.e. the discovery of splice variants of “at risk” transcripts, we can identify presymptomatic individuals on the basis of their possessing an abnormal ratio of spliced isoforms from a single gene. Such detection is increasingly more important today as new neuroprotective agents are developed and can be used to combat disease in early stages. Furthermore, the elucidation of altered gene products sets the stage for the development of novel therapeutic strategies to target specific splicing isoforms.

Alternative Precursor-Messenger RNA (Pre-mRNA) Splicing

Pre-mRNA splicing is a regulated process to remove non-coding sequences called introns from the pre-mRNA. It occurs after gene transcription and prior to mRNA translation. Alternative pre-mRNA splicing is the process of differential inclusion or exclusion of regions of the pre-mRNA that allows a single transcript to produce one or more variants of RNA and protein products. While alternative splicing for many genes is found in normal individuals, alternative or regulated splicing may be the cause of pathogenic changes at RNA and protein levels. Alternative splice products have been identified in certain diseases, including experimental and clinical Parkinson's disease. In the present disclosure, we show that the changes in the ratios of splice variants of certain genes, such as FosB, are indicative of neurodegenerative disease.

Neurological Disease is Linked to Aberrant Splicing

Immediately after the discovery of RNA splicing, it was apparent that mistakes in and dysregulation of RNA processing could lead to disease. The magnitude of the contribution of such problems is only beginning to be understood. In order to understand how mistakes in splicing and alteration of its regulation leads to disease, it was necessary to first identify the essential components and characterize their function. It is clear that proper splicing in humans requires the recognition of weakly conserved splice sites at the 5′ and 3′ splice sites and the branch-point of the lariat intermediate. The identification of exons and introns involves five small ribonucleoprotein particles (snRNPs) and eighty or more proteins. Because the recognitions sequences for splicing are loosely defined, additional sequences referred to as enhancers and silencers are also required. There are several factors that regulate splicing by binding to these recognition sequences. The concentration, distribution, composition and state of modification of these regulatory factors determine whether they enhance or inhibit a particular splice site.

While some cases of neurodegenerative diseases are likely to be a result of genetic mutation, these cases are rare. The majority of cases of Alzheimer's disease (AD), the most common neurodegenerative disease, and Parkinson's disease (PD), the most common neurodegenerative movement disorder, are idiopathic. At least 90% of the cases of amyotrophic lateral sclerosis (ALS) are also sporadic. Since the vast majority of the cases of neurodegenerative disease are sporadic, it would be beneficial to determine the underlying mechanisms in order to identify biomarkers to characterize disease progression and to design treatments.

The main problem with determining the basis of the sporadic forms of neurodegenerative disease is distinguishing which molecular changes are primary events and which are secondary. One thing which is known, however, is that oxidative stress and exposure to heavy metals, along with other cellular stresses, may cause a dysregulation (or loss of proper regulation) of splicing that alters the ratio of splice variants and produces disease. There are many known examples of neurodegeneration resulting from dysregulation of splicing. The loss of proper regulation is most likely due to changes in the regulatory factors brought about by environmental stimuli. It is likely that mistakes in splicing and a loss of its regulation is a common mechanism underlying neurodegenerative disease.

Many cancers and inherited diseases are associated with abnormalities in the regulation of splicing. There are numerous examples of changes in splicing associated with inherited neurological diseases (D'Souza et al., 1999, Missense and silent tau gene mutations cause frontotemporal dementia with parkinsonism-chromosome 17 type by affecting multiple alternative RNA splicing regulatory elements. Proc Natl Acad Sci USA 96:5598-5603; Grabowski and Black, 2001, Alternative RNA splicing in the nervous system. Prog Neurobiol 65:289-308; Zhang et al., 2002, Region-specific alternative splicing in the nervous system: implications for regulation by the RNA-binding protein NAPOR. RNA 8:671-685). However, it is unclear how splicing regulation becomes disrupted in patients who do not inherit genetic defects. Some patients may acquire a genetic defect that affects splicing after exposure to environmental stimuli. It is also possible in other cases that exposure to stressful environmental stimuli activates new signaling pathways that result in a change in the concentration, localization, and/or modification of regulatory factors required for the maintenance of proper splicing. There are numerous examples of altered splice site selection in response to stresses such as pH change, osmotic or temperature shock, exposure to UV light or forced physical activity (see Stamm, 2002, Signals and their transduction pathways regulating alternative splicing: a new dimension of the human genome. Hum Mol Genet 11:2409-2416). One example is the translocation of SR regulatory factors from the nucleus to the cytoplasm in brain ischemia (Daoud et al., 2002, Ischemia induces a translocation of the splicing factor tra2-beta 1 and changes alternative splicing patterns in the brain. J Neurosci 22:5889-5899). The result of this change is a disruption of the normal regulated splicing of the interleukin-1β converting enzyme homologue 1 pre-mRNA. Similarly, an increase in the intracellular calcium concentration may result in a translocation of splicing factors. Based on the very limited number of studies addressing this problem, it is obvious that further research is needed to elucidate how splicing changes are initiated by environmental factors. The present invention discloses the gene expression changes that occur in mammalian subjects with PD, and possibly other neurodegenerative diseases, in which the ratio of one mRNA splice product to another mRNA splice product is altered. The amounts of mRNAs of these splice variants in a subject can be used as biomarkers of the disease process for neurodegenerative diseases such as PD. For idiopathic neurodegeneration, splice variants of RNA are better biomarkers than specific genes since it represents the molecular changes in gene expression responsible for the disease development when no genomic mutations are present.

Alternatively Spliced Transcripts Whose Expression Changes in Models of PD.

Some changes in gene expression that occur in animals and cell culture models of PD have been identified (Youdim et al., 2002, Early and late molecular events in neurodegeneration and neuroprotection in Parkinson's disease MPTP model as assessed by cDNA microarray; the role of iron. Neurotox Res 4:679-689; Mandel et al., 2003, Using cDNA microarray to assess Parkinson's disease models and the effects of neuroprotective drugs. Trends Pharmacol Sci 24:184-191). In these studies, cDNA microarrays are often used to identify an increase or decrease in RNA expression. However, the technique does not provide information about post-transcriptional modifications and thus microarrays only provide a partial view of the biological changes that occur in disease development.

We investigated whether transcripts that either increased or decreased in expression in these studies have alternatively spliced products. We used data from the putative alternative splice database and published results. Our search indicated that approximately 50% of the transcripts whose expression was increased or decreased in parkinsonism or in PD may have splice variants. Some of these transcripts are listed in Table 1.

Splice variants of α-synuclein, synphilin, syntaxin 8, parkin, FosB, RGS9-2 and Nurr1 are especially of interest since these genes have been associated with Parkinsonism in animals or PD in patients. In one study which investigated the expression of FosB and the Regulator of G protein signaling 9 (RGS9) in human PD patients, it was found that both RGS9-2 and ΔFosB protein levels were elevated in the striatum of these patients (Tekumalla P. K. et al., 2001, Elevated levels of DeltaFosB and RGS9 in striatum in Parkinson's disease. Biol Psychiatry 50:813-816). RGS9-2 and ΔFosB are the products of alternative splicing. The study, however, did not look at the FosB or RGS9 mRNAs.

TABLE 1
Gene Expression Data.
Selected transcripts that have splice variants appear in the table.
A total of 98 oxidative stress (OS), 27 MPTP (mptp), 11
Methamphetamine (M), and 1 cocaine-associated transcripts have
been found to be altered in microarray studies and studies on
PD patients and animal models of Parkinsonism.

| Function | Name | Study | Change |
|---|---|---|---|
| Stress | junD | M | Up |
| | Gtase | mptp | Down |
| | junB | M | Up |
| | HemeOxygenase 1 | OS | Up |
| Receptors | IL-1 recept II | mptp | Up |
| | IL-2 rp-G | mptp | Up |
| | IL-10 | mptp | Up |
| | Ach N receptor | mptp | Up |
| ER/ubiquitin-like | U-like protein3 | OS | Up |
| | Torsin | OS | Up |
| Splicing | U2AF1-RS1 | M | Down |
| Cell cycle | Cyclin 82 | mptp | Down |
| | C/EBP | M | Up |
| Apoptosis | BAX M, isoform-a | mptp, M | Up |
| | c-Myc | M | Up |
| | Apaf1 | OS | Up |
| Protease | Cathepsin H | M | Up |
| PD-associated | Alpha-synuclein | mptp | Up |
| | FosB | Cocaine | Up |
| | RGS9-2 | PD | unknown |
| | Nurr1 | PD | unknown |
| | Synphilin | PD | unknown |
| | Parkin | PD | unknown |
| | Syntaxin 8 | PD | unknown |

ΔFosB and FosB

The FosB protein is one of the four members of the Fos family, which also includes c-Fos, Fra-1 and Fra-2. Fos proteins are components of the activator protein-1 (AP-1) family of transcription factors that bind DNA at specific promoter or enhancer regions or sites, where they regulate transcription. These Fos family proteins heterodimerize with Jun family proteins (c-Jun, JunB, or JunD) to form active AP-1 transcription factors that bind to AP-1 sites present in the promoters of certain genes to regulate their transcription. These Fos family proteins are induced rapidly and transiently in specific brain regions after acute administration of many drugs of abuse. These proteins return to basal levels within hours of administration.

The nucleotide sequences of the fosB gene share many similarities among various mammalian species. In the human, the nucleotide sequence of the fosB gene is shown in SEQ ID NO:1 in which nucleotides 4557-4712 (SEQ ID NO:2) represent exon 4 and 4853-7184 (SEQ ID NO: 3) represent exon 5.

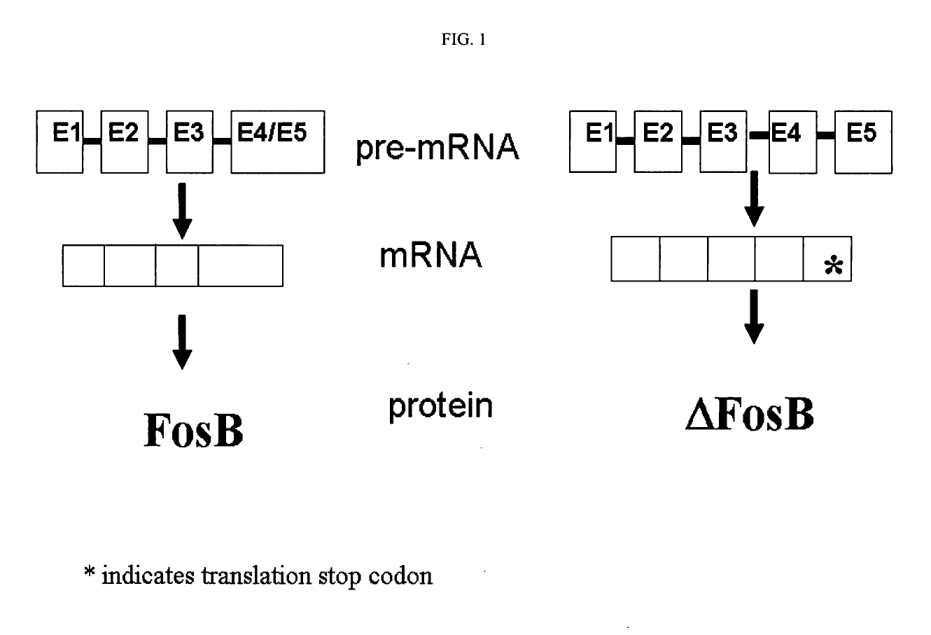

ΔFosB (MW 35-37kDa) is an isoform of the FosB protein encoded by the fosB gene that is produced by alternative splicing of the FosB pre-mRNA. While the FosB pre-mRNA is spliced to remove three introns to form the FosB mRNA, the ΔFosB mRNA is formed by splicing of the FosB pre-mRNA to remove four introns (see FIG. 1). When four introns are removed, an open-reading-frame shift occurs and a translation stop codon is produced. This results in the production of a truncated ΔFosB protein that is missing the carboxy-terminus.

ΔFosB and FosB Transcripts as an Indication of a Neurodegenerative Disease

The present invention provides a method of diagnosing a neurodegenerative disease in a mammalian subject, preferably human. The method comprises obtaining RNA from the mammalian subject, and assaying the RNA for an increase in amount of ΔFosB mRNA or the ratio of the amount of ΔFosB mRNA to the amount of FosB mRNA as compared to that of a control. An increase in the amount of ΔFosB mRNA or the ratio of the amount of ΔFosB mRNA to the amount FosB mRNA as compared to that of the control is indicative of the presence of the neurodegenerative disease.

The present invention further provides a method of prognosticating a neurodegenerative disease in a mammalian subject, preferably a human subject. The method comprises obtaining RNA from the subject over time, and assaying the RNA for the amount of ΔFosB and FosB mRNAs. An increase in the amount of ΔFos mRNA or the ratio of the amount of ΔFosB mRNA to the amount of FosB mRNA is indicative of a negative prognosis, whereas no change in the amount of ΔFosB mRNA or the ratio of the amount of ΔFosB mRNA to the amount of FosB mRNA or a decrease in the amount of ΔFosB mRNA or the ratio of the amount of ΔFosB mRNA to the amount of FosB mRNA is indicative of a positive prognosis.

The present invention still further provides a method of assessing the efficacy of treatment of a neurodegenerative disease in a mammalian subject in need of the treatment. The subject is preferably a human subject. The method comprises obtaining RNA from the subject during the course of treatment, and assaying the RNA for the amounts of ΔFosB and FosB mRNAs. An increase in the amount of ΔFosB mRNA or the ratio of ΔFosB mRNA to the amount of FosB mRNA in the RNA indicates that the treatment is ineffective, whereas no change in the amount of ΔFosB mRNA or the ratio of the amount of ΔFosB mRNA to the amount of FosB mRNA or a decrease in the ratio of the amount of ΔFosB mRNA to the amount of FosB mRNA indicates that the treatment is effective.

The present invention yet further provides a method of screening potential therapeutic agents for the treatment of a neurodegenerative disease in a mammalian subject in need of the treatment. The subject is preferably a human subject. The method comprises administering the therapeutic agent to be screened to the subject, obtaining RNA from the subject, and assaying the RNA for the amounts of ΔFosB and FosB mRNAs. An increase in the amount of ΔFosB mRNA or the ratio of the amount of ΔFosB mRNA to the amount of FosB mRNA indicates that therapeutic agent being screened is a potential therapeutic agent for the treatment of the neurodegenerative diseases, whereas no change in the amount of ΔFosB mRNA or the ratio of the amount of ΔFosB mRNA to the amount of FosB mRNA or a decrease in the ratio of the amount of ΔFosB mRNA to the amount of FosB mRNA indicates that the therapeutic agent being screened is not a potential therapeutic agent in the treatment neurodegenerative diseases.

Any neurodegenerative disease resulting in an increase in the ΔFosB mRNA or the ratio of the amount of ΔFosB mRNA to the amount of FosB mRNA can be diagnosed or prognosticated in accordance with the above methods of diagnosis and prognosis. Likewise, the efficacy of treatment of any such neurodegenerative disease can be assessed in accordance with the above method of assessing the efficacy of treatment. Examples of neurodegenerative diseases include, but are not limited to, Parkinson's disease and Alzheimer's disease.

RNA can be obtained from the tissue, such as but is not limited to blood and cerebrospinal fluid (CSF), of a mammalian subject, particularly human, using any suitable method. Preferably, venous blood or CSF is drawn in accordance with methods known in the art. Preferably, the blood or cerebrospinal fluid is kept cold, e.g., on ice, until use. When using plasma, blood should not be permitted to coagulate prior to separation of the cellular and acellular blood components. Preferably, within 30 min of drawing blood, serum is separated by centrifugation, e.g., at 1100×g at 4° C. Serum or plasma can be frozen, for example, at −70° C., after separation from the cellular component of blood. When using frozen blood plasma or serum, the frozen plasma or serum can be slowly thawed or rapidly thawed, for example in a water bath at 37° C., and RNA is extracted without delay.

RNA can be extracted from the blood or CSF using any suitable method, such as the methods set forth below in Example 2. Desirably, the RNA is extracted as soon as possible so as to minimize degradation of the RNA. Examples of suitable extraction methods include, but are not limited to, gelatin, silica, glass bead, diatom, guanidinum thiocyanate acid-phenol, guanidinium thiocyanate acid, centrifugation through cesium chloride or a similar gradient, phenol-chloroform, or a commercially available kit, such as the Promega SV total RNA isolation system (Promega, Madison, Wis.), the Perfect RNA Total RNA Isolation Kit (Five Prime-Three Prime, Inc., Boulder, Colo.), or the TRI Reagent BD kit (Molecular Research Center, Inc., Cincinnati, Ohio). Alternatively, extraction can be performed using probes that specifically hybridize to a particular RNA, in particular isolation methods dependent thereupon, e.g., chromatographic methods and methods for capturing RNA hybridized to the probes.

The extracted RNA is then amplified, either after conversion into cDNA or directly, using in vitro amplification methods. Examples of amplification methods include, but are not limited to, reverse transcriptase-polymerase chain reaction (RT-PCR; see, e.g., U.S. Pat. Nos. 4,683,195; 4,683,202; and 4,800,159 for PCR techniques, generally), ligase chain reaction, DNA signal amplification, amplifiable RNA reporters, Q-beta replication, transcription-based amplification, boomerang DNA amplification, strand displacement activation, cycling probe technology, isothermal nucleic acid sequence-based amplification, and other self-sustained sequence replication assays.

Preferably, the RNA is amplified using RT-PCR. RNA can be reverse-transcribed into cDNA using Moloney murine leukemia virus (MMLV) reverse transcriptase (Promega), a reaction buffer supplied by the manufacturer, dNTPs, random hexameric oligonucleotide primers, and RNAsin (Promega). Reverse transcripton is typically performed by incubation at room temperature for 10 min, followed by incubation at 37° C. for 1 hr. Alternatively, reverse transcription can be performed in accordance with the method of Rajagopal et al. Epidermal growth factor expression in human colon and colon carcinomas: anti-sense epidermal growth factor receptor RNA down-regulates the proliferation of human colon cancer cells Int. J Cancer 62: 661-667) or Dahiya et al., 1996 (Differential gene expression of transforming growth factors alpha and beta, epidermal growth factor, keratinocyte growth factor, and their receptors in fetal and adult human prostatic tissues and cancer cell lines, Urology 48: 963-970). Amplification oligonucleotide primers are selected to be specific for amplifying the nucleic acid of interest. Therefore, the oligonucleotide primers need to be of sufficient length to achieve specificity, yet not so long as to affect adversely the efficiency of the reaction. Optimally, the primers are from about 18 to about 21 nucleotides in length. While primers derived from rat and mouse FosB sequences can be used (SEQ ID NOS:4 and 5), it is desirable to use primers derived from human FosB sequences (Martin-Gallardo et al., 1992, Automated DNA sequencing and analysis of 106 kilobases from human chromosome 19q13.3, Nat. Genet. 1(1): 34-39). Any sequence from exon 4, such as SEQ ID NO:2 (nucleotides 4557-4712 in SEQ ID NO:1), that is of sufficient length, e.g., 18-21 nucleotides, can be used as a sense primer. Likewise, any sequence from exon 5, such as SEQ ID NO:3 (nucleotides 4853-7184 in SEQ ID NO:1), that is of sufficient length, e.g., 18-21 nucleotides, can be used as an antisense primer. Preferably, the antisense primer is obtained within nucleotides 4853-6000 in SEQ ID NO:1. Examples of preferred sense and antisense primers are those that correspond to SEQ ID NO: 6 (nucleotides 4581-4598 in SEQ ID NO:1, aaagcagagctggagtcg) and SEQ ID NO: 7 (nucleotides 4882-4901 in SEQ ID NO:1, gtacgaagggttaacaacgg), respectively. Other examples of suitable primers include, but are not limited to, those set forth in Example 1, such as SEQ ID NO:4 (sense primer) and SEQ ID NO:5 (antisense primer).

The amplified product then can be separated, such as by gel electrophoresis (e.g., 2% agarose gel), and visualized (e.g., ethidium bromide staining) in accordance with methods known in the art. Alternatively, capillary electrophoresis, amplification using biotinylated or otherwise modified primers, nucleic acid hybridization using specific, detectably-labeled probes, such as fluorescently, radioactively, or chromogenically labeled probes, Southern blot, Northern blot, electrochemiluminescence, laser-induced fluorescence, reverse dot blot, or high performance liquid chromatography can be used. Detection can be qualitative or quantitative.

The methods disclosed in the present invention can be performed using a kit. The kit can comprise oligonucleotide primers specific for cDNA synthesis, alone or in further combination with reagents for reverse-transcribing RNA into cDNA, reagents for in vitro amplification, and/or reagents for RNA extraction. For example, the kit can comprise sense and antisense primers for human FosB, such as primers that are about 18 to about 21 nucleotides in length. Examples of preferred sense and antisense primers are those that correspond to SEQ ID NO:6 (nucleotides 4580-4598 in SEQ ID NO:1, aaagcagagctggagtcg) and SEQ ID NO:7 (nucleotides 4882-4901 in SEQ ID NO:1, gtacgaagggttaacaacgg), respectively. Other examples of suitable primers include, but are not limited to, those set forth in Example 1, such as SEQ ID NO:4 (sense primer) and SEQ ID NO:5 (antisense primer).

### EXAMPLES

The following examples serve to illustrate the present invention and are not intended to limit its scope in any way.

### Example 1

This example demonstrates that alternative splicing of FosB RNA transcripts is associated with Parkinsonism and progressive cell death.

The chronic 1-methyl-4-phenyl-1,2,3,6-tetrahydropyridine (MPTP)/probenecid mouse model was used (Petroske et al., 2001, Mouse model of parkinsonism: A comparison between subacute MPTP and chronic MPTP/probenecid treatment, Neuroscience 106(3): 589-601). Exposure to MPTP causes Parkinsonism in humans and other species. It is converted to the pyridium ion MPP⁺ by the glia and is selectively uptaken by the dopamine (DA) neurons in the substantia nigra. MPP⁺ is a potent complex I inhibitor which interferes with mitochondrial respiration which results in cell death. When multiple and high doses of MPTP are administered to C57BL mice, the MPP⁺ ion converted from MPTP selectively destroys the DA neurons to significantly deplete dopamine. The extracellular concentration of the MPP⁺ ion can be prolonged in the presence of the adjuvant probencid, which reduces the excretion of MPTP from the brain and kidney. Combined MPTP and probenecid treatment provides us with a chronic mouse model suitable for studying PD. This chronic model replicates the symptoms of the disease over a long period of time.

C57BL/6 mice (Charles River Laboratories, Wilmington, Mass.), which were 8-10 weeks old, were used. Mice were housed two per cage with free access to food and water. The room was maintained at constant temperature and humidity on a 12-hour light-dark cycle. Mice were injected subcutaneously with MPTP hydrochloride (25 mg/kg in saline) in combination with probenecid (250 mg/kg in dimethyl sulfoxide) every 3.5 days for a total of 10 doses. Control mice received 10 doses of probenecid or saline alone.

After one to two weeks of administration of MPTP/probenecid, approximately 50% of DA neurons are destroyed. At this stage, there is no accumulation of α-synuclein in midbrain cells and motor deficits are not yet evident. By about three weeks post administration of MPTP/probenecid, more than 70% of DA neurons are destroyed, sporadic aggregations of cytosolic α-synuclein occurs and motor deficit is apparent. These pathophysiological features persist for about 6 months post-treatment.

Mice treated with MPTP/probenecid for 5 weeks show significant depletion of dopamine in the stiratum after the last treatment as compared to the controls which receive only the vehicle. Theres is also a significant loss of TH-immuoreactive neurons in these MPTP/probenecid treated mice at week 5 and week 24 after the last treatment as compared to controls.

Behavioral assessment of the mice indicated that the MPTP/probenecid-treated mice exhibited impaired locomotor performance compared to matched controls (p<0.05) in a grid test. Mice were placed on a grid, and the grid was then rotated upside down. Mice exhibiting parkinsonian symptoms lost forepaw grip on the grid. Forepaw faults per total movements were determined for MPTP/probenecid-treated mice and control mice to determine locomotive impairment. The results of the behavioral assessment indicated that the locomotive ability of the mice was impaired after chronic MPTP/probenecid treatment.

The striatum, substantia nigra with some surrounding tissue, and cortex were removed from mice brains and put on ice. Total RNA was extracted using the Promega SV total RNA isolation system (Promega; Catalog No. Z3100). Brain tissue was sonicated in SV RNA lysis buffer (30 mg tissue per 175 μl of buffer) and mixed with 350 μl of SV RNA dilution buffer. The solution was then centrifuged for 10 min at 14,000×g. The supernatant was transferred to a fresh microcentrifuge tube, mixed with 200 μl of 95% ethanol and applied to a spin column. The column was centrifuged for 10 min at 14,000×g and then washed twice with SV RNA wash solution. DNase I (5 μl) in 40 μl of Yellow Core Buffer and 5 μl of 0.09 M MnCl₂ was applied to the column and incubated at room temperature for 15 min, and then 200 μl of DNase Stop Solution were added to each sample. The samples were then washed twice with 900 μl of SV RNA Wash Solution. RNA was eluted in 100 μl of nuclease-free water. The RNA was aliquoted and stored at −80° C.

Total RNA was reverse-transcribed in 20 μl reactions containing 1 mM dNTP mix, 0.5 μg of random primers, 1 μg of total RNA, and 0.5 μl of AMV reverse transcriptase (10 U/μl; Promega) in reverse transcription buffer (10 mM Tris-HCl (pH 9.0), 50 mM KCl, and 0.1% Triton X-100). Reactions were incubated for 2 hrs at 37° C.

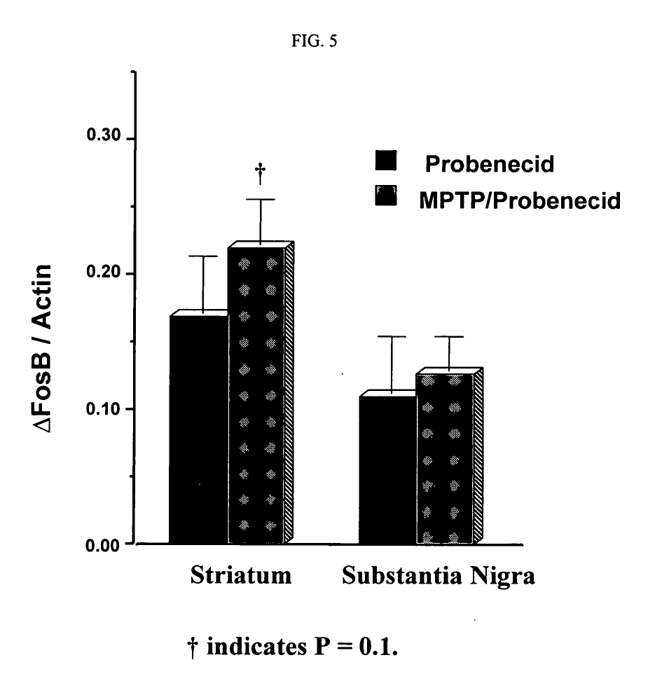

Two primers that recognize FosB exon 4 and exon 5 were used to amplify the cDNA. The primers corresponded to mouse sequences 5520-5539 (SEQ ID NO:4) and 5823-5842, (SEQ ID NO:5) which are identical to the rat sequences (Lazo et al., 1992, Structure and mapping of the fosB gene. FosB downregulates the activity of the fosB promoter, Nucleic Acids Res. 20:243-350). The PCR (20 μl) reactions contained 1 μl of cDNA, 20 pmol of each primer, 1 μl Taq polymerase (5 U/μl; Promega), 1 mM dNTP mix in 1.5 mM MgCl₂, 10 mM Tris-HCl (pH 9.0), 50 mM KCl, and 0.1% Triton X-100. The cycling conditions used for the amplification were 2 min at 94° C., 15 sec at 94° C., 30 sec at 60° C., and 30 sec at 68° C. for 35 cycles, then 2 min at 68° C. and overnight at 4° C. Full-length FosB and ΔFosB products were resolved on a 2% agarose gel stained with ethidium bromide. The results are shown in FIGS. 5 to 7.

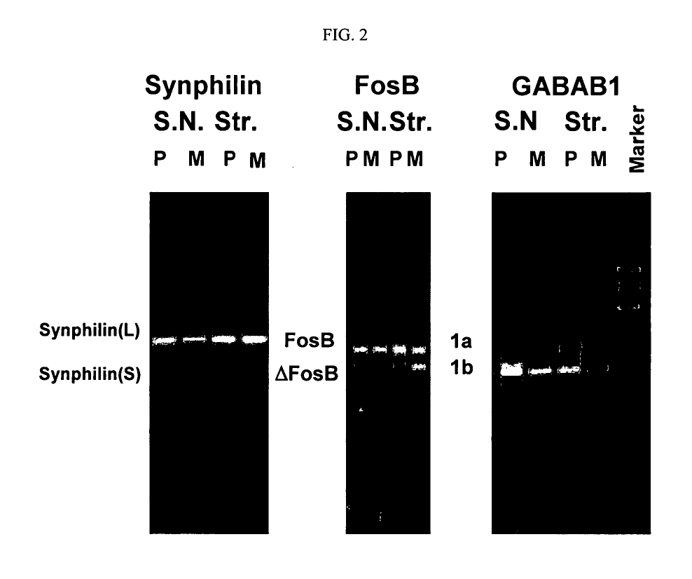

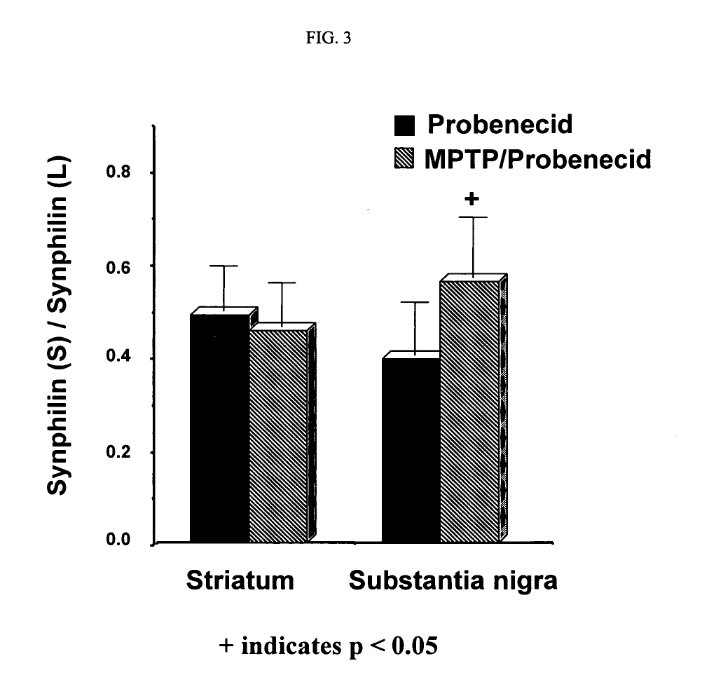

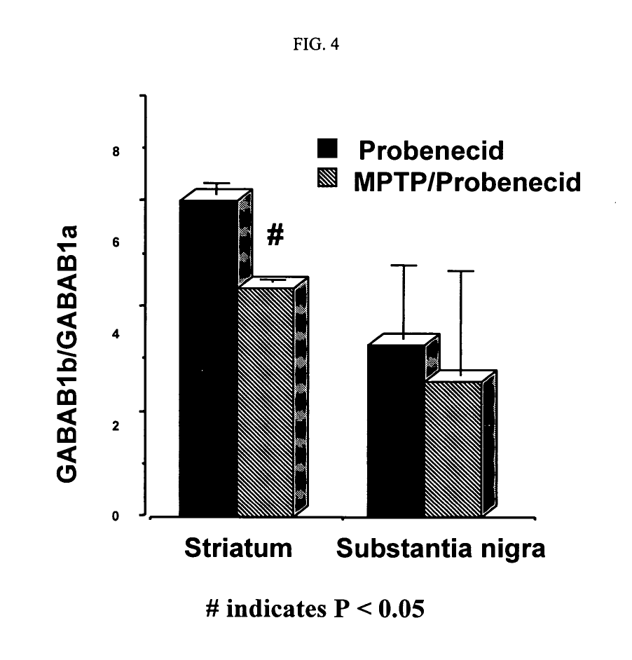

FIG. 2 shows the representative images of PT-PCR amplification of Synphilin, FosB and GABAB1 receptor RNA variants. The ratio of small to large synphilin RNA increased in the substantia nigra of MPTP/probenecid-treated animals, but did not change in the striatum (FIG. 3). The ratio of GABAB1b to GABAB1a RNAs decreased in the striatum of MPTP/probenecid-treated animals, but not in the substantia nigra (FIG. 4).

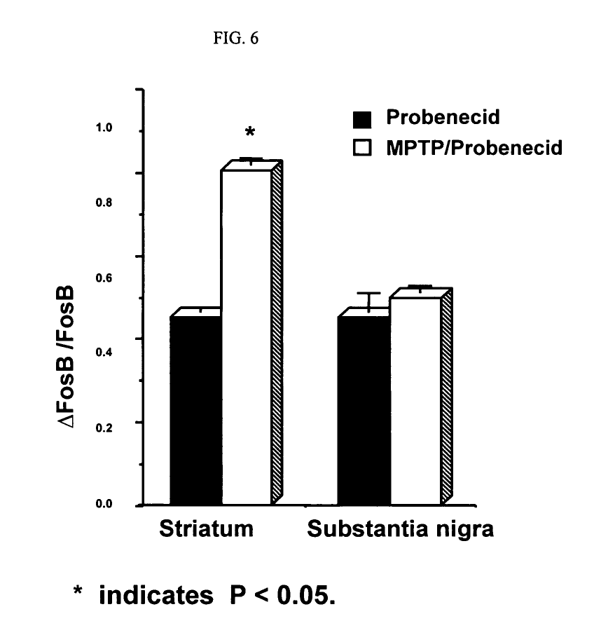

Although ΔFosB protein increased only slightly (P=0.1) in the striatum of mice after MPTP/probenecid treatment using actin as the loading control (FIG. 5),the ratio of small FosB (ΔFosB) mRNA to large FosB mRNA increased significantly in the striatum of MPTP/probenecid-treated animals (FIG. 6), but not in the substantia nigra or cortex. The data indicate that the regulation of pre-mRNA splicing of the FosB gene changes with parkinsonism and the change in the alternate spliced mRNA is a better indication of parkinsonism than the protein itself. It is anticipated that similar changes in alternative splicing occur in other tissues such as the blood or the cerebrospinal fluid (CSF) of PD patients, as is the case in the Alzheimer's disease patients in which it was demonstrated that acetylcholinesterase splice variants were found both in the parietal cortex as well as the CSF (T. Darrch-Shori et. al., 2004, Long-lasting acetylcholinesterase splice variants in anticholinesterase-treated Alzheimer's disease patients, J. Neurochem., 88:1102-1113). In addition, alternative splice variants of acetylcholinesterase were found both in brain neurons and peripheral blood cells in stress-induced laboratory animals (M. Pick et al., 2004, From Brain to Blood: Alternative Splicing Evidence for the Cholinergic Basis of Mammalian Stress Responses, Ann. N.Y. Acad. Sci, 1018: 85-94).

### Example 2

This example describes the preparation of RNA from blood and CSF.

Blood and CSF samples are kept frozen at −70° C. until assayed. Blood samples are processed using the Promega SV Total RNA Isolation System. Blood (1 ml) is collected in heparinized tubes and centrifuged at 400×g. The supernatant is removed from the pellet and placed in a separation test tube to be processed as described below. Red Blood Cell Lysis Solution (1 ml of 4 M guanidinium thiocyanate (GTC), 0.01 M Tris (pH 7.5) and 0.97% β-mercaptoethanol) is added to the pellet, and the cells are resuspended by gentle pipetting. The lysed cells are centrifuged at 3,000 rpm for 5 min. Supernatant (1 ml) is removed from the top of this tube and discarded. Red Blood Cell Lysis Solution (1 ml) is added to the remaining sample. Resuspension, centrifugation and disposal of the top layer are repeated twice more. With each repetition, the size of the pelleted material decreases. Upon the final repetition, all but 100 μl of supernatant is removed from the pellet and discarded. SV RNA Lysis Buffer (175 μl) is added to the remaining sample and resuspended by pipetting. SV RNA Dilution Buffer (350 μl) is added to this sample and mixed by inverting the test tube several times. The sample is incubated at 70° C. for a maximum of 3 min. The sample is centrifuged at 14,000×g for 10 min at room temperature. The cleared lysate is transferred to a fresh test tube and 95% ethanol (200 μl) is added. The sample is then transferred to a spin column and processed as described for brain tissue.

Blood supernatant, cleared of cells by centrifugation, and CSF can be processed using the Tri Reagent LS kit (Sigma, St. Louis, Mo.). If the sample volume is less than 0.25 ml, the volume can be brought up to 0.25 ml by the addition of distilled water. Tri Reagent LS (0.75 ml) is added to the sample, and the sample is mixed by pipetting and then incubated for 5 min at room temperature. 1-bromo-3-chloropropane (0.1 ml) is added to each sample, and the samples are vortexed for 15 sec and then allowed to stand at room temperature for 2-15 min. Samples are then centrifuged at 12,000×g for 15 min at 4° C. The upper, colorless phase contains RNA. This phase is transferred to a fresh tube, and RNA is precipitated by the addition of 0.5 ml of isopropanol. The samples are incubated at room temperature for 10 min and then centrifuged at 12,000×g for 8 min at 4-25° C. The RNA pellet is washed with 1 ml of 75% ethanol and centrifuged again at 7,500×g for 5 min at 4-25° C. The pellet is partially air dried and then resuspended in reverse transcription buffer as described above. It is anticipated that the amount of ΔFosB mRNA or the ratio of the amount of ΔFosB mRNA to the amount of FosB mRNA is increased in PD animal models or patients with PD.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

It is understood that, given the above description of the embodiments of the invention, various modifications may be made by one skilled in the art. Such modifications are intended to be encompassed by the claims below.

What is claimed is:

1. A method of diagnosing a neurodegenerative disease in a mammalian subject comprising:
(i) obtaining RNA from the subject, and
(ii) assaying the RNA for an increase in the amount of ΔFosB mRNA or an increase in the ratio of the amount of ΔFosB mRNA to the amount of FosB mRNA as compared to a control,
wherein an increase in the amount of ΔFosB mRNA or an increase in the ratio of the amount of ΔFosB mRNA to the amount of FosB mRNA as compared to the control is indicative of the presence of the neurodegenerative disease.

2. The method of claim 1, wherein the mammalian subject is a human subject.

3. The method of claim 1, wherein the neurodegenerative disease is Parkinson's disease.

4. The method of claim 1, wherein the neurodegenerative disease is Alzheimer's disease.

5. The method of claim 1, wherein the RNA is obtained from blood from the subject.

6. The method of claim 1, wherein the RNA is obtained from cerebrospinal fluid from the subject.

7. A method of prognosticating a neurodegenerative disease in a mammalian subject comprising:
(i) obtaining RNA from the subject over time, and
(ii) assaying the RNA for the amount of ΔFosB mRNA and FosB mRNA,
wherein an increase in the amount of ΔFosB mRNA or an increase in the ratio of the amount of ΔFosB mRNA to the amount of FosB mRNA is indicative of a negative prognosis, and
wherein no change or a decrease in the amount of ΔFosB mRNA or no change or a decrease in the ratio of the amount of ΔFosB mRNA to the amount of FosB mRNA is indicative of a positive prognosis.

8. The method of claim 7, wherein the subject is a human subject.

9. The method of claim 7, wherein the neurodegenerative disease is Parkinson's disease.

10. The method of claim 7, wherein the neurodegenerative disease is Alzheimer's disease.

11. The method of claim 7, wherein the RNA is obtained from blood from the subject.

12. The method of claim 7, wherein the RNA is obtained from cerebrospinal fluid from the subject.

13. A method of assessing the efficacy of treatment of a neurodegenerative disease in a mammalian subject comprising:
(i) obtaining RNA from the subject during the course of treatment, and
(ii) assaying the RNA for the amount of ΔFosB mRNA and FosB mRNA,
wherein an increase in the amount of ΔFosB mRNA or an increase in the ratio of the amount of ΔFosB mRNA to the amount of FosB mRNA indicates that the treatment is ineffective, and
wherein no change or a decrease in the amount of ΔFosB mRNA or no change or a decrease in the ratio of the amount of ΔFosB mRNA to the amount of FosB mRNA indicates that the treatment is effective.

14. The method of claim 13, wherein the mammalian subject is a human subject.

15. The method of claim 13, wherein the neurodegenerative disease is Parkinson's disease.

16. The method of claim 13, wherein the neurodegenerative disease is Alzheimer's disease.

17. The method of claim 13, wherein the RNA is obtained from blood from the subject.

18. The method of claim 13, wherein the RNA is obtained from cerebrospinal fluid from the subject.

19. A method of screening a therapeutic agent for potential treatment of a neurodegenerative disease in a mammalian subject in need of the treatment comprising:
(i) obtaining RNA from the subject during or after administrating the therapeutic agent to be screened to the subject, and
(ii) assaying the RNA for the amount of ΔFosB mRNA and FosB mRNA,
wherein an increase in the amount of ΔFosB mRNA or an increase in the ratio of the amount of ΔFosB mRNA to the amount of FosB mRNA indicates that the therapeutic agent is a potential agent for treating the neurodegenerative disease, and
wherein no change or a decrease in the amount of ΔFosB mRNA or no change or a decrease in the ratio of the amount of ΔFosB mRNA to the amount of FosB mRNA indicates that the therapeutic agent is not a potential agent for treating the neurodegenerative disease.

20. The method of claim 19, wherein the mammalian subject is a human subject.

21. The method of claim 19, wherein the neurodegenerative disease is Parkinson's disease

22. The method of claim 19, wherein the neurodegenerative disease is Alzheimer's disease.

23. The method of claim 19, wherein the RNA is obtained from blood from the subject.

24. The method of claim 19, wherein the RNA is obtained from cerebrospinal fluid from the subject.

25. A kit for use in diagnosing or prognosticating a neurodegenerative disease or in screening therapeutic agents for treating a neurodegenerative disease in a mammalian subject, which kit comprises a sense primer from a nucleotide sequence in exon 4 of the human or mouse FosB gene and an antisense primer from a nucleotide sequence in exon 5 of the human or mouse FosB gene.

26. The kit of claim 25, wherein the sense primer is selected from the group consisting of SEQ ID NO:2, SEQ ID NO:4 and SEQ ID NO:6.

27. The kit of claim 25, wherein the sense primer is SEQ ID NO:6

28. The kit of claim 25, wherein the antisense primer is selected from the group consisting of SEQ ID NO:3, SEQ ID NO:5 and SEQ ID NO:7.

29. The kit of claim 25, wherein the antisense primer is SEQ ID NO:7.

30. The kit of claim 25, wherein each of the sense primer and the antisense primer is about 18-21 nucleotides in length.

31. The kit of claim 25, wherein the mammalian subject is a human subject.

32. A method for diagnosing a neurodegenerative disease in a mammalian subject comprising monitoring the subject for one or more alternatively spliced pre-mRNA transcripts of genes that produce splice variants in neurodegenerative diseases or genes with altered expressions in neurodegenerative diseases, wherein a change in the amount of one or more of the alternatively spliced pre-mRNA transcripts is indicative of the presence of the neurodegenerative disease.

33. The method of claim 31, wherein the change is an increase or a decrease in the amount of the alternatively spliced pre-mRNA transcripts or an increase or a decrease in the ratio of the amount of the alternatively spliced pre-mRNA transcript to the amount of the corresponding normal spliced pre-mRNA transcript.
